(19) United States
(12) Patent Application Publication
(10) Pub. No.: US 2006/0252773 A1
(43) Pub. Date: Nov. 9, 2006

(54) Method for the treatment of drug abuse

(75) Inventor: Angelo Ceci, Mittelbiberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/381,130

(22) Filed: May 2, 2006

(30) Foreign Application Priority Data
May 6, 2005 (EP) 05009922

(51) Int. Cl. A61K 31/496

(52) U.S. Cl. 514/254.06

(57) ABSTRACT

The invention relates to a method for the treatment of drug addiction, especially nicotine, ethanol and psychostimulants addiction, and for changing dependency-related behaviors of a person suffering from substance addiction in the course of such treatment, comprising the administration of a therpeutically effective amount of flibanserin.

The invention relates to a method for the treatment of drug addiction, especially nicotine, ethanol and psychostimulants addiction, and for changing dependency-related behavior of a person suffering from substance addiction comprising the administration of a therpeutically effective amount of flibanserin.

### DESCRIPTION OF THE INVENTION

Substance addiction, such as drug abuse, and the resulting addiction-related behavior are enormous social and economic problems that continue to grow with devastating consequences.

A very frequently abused drug is nicotine. This drug is present e.g. in cigars, cigarettes, chewing tobacco, snuff and other tobacco products. It is generally known that nicotine contributes to various diseases like heart disease, respiratory disease or cancer. It is known that the discontinuation of tobacco abuse results in accompaniments like irritability, anxiety, restlessness, lack of concentration, lightheadedness, insomnia, tremor, increased hunger and weight gain, and, of course, an intense craving for tobacco.

Ethanol is probably the most abused drug and a major cause of morbidity and mortality. Frequent consumption of large amounts of ethanol affects nearly every organ system in the body, e.g. the gastrointestinal tract (gastritis, stomach ulcers, duodenal ulcers, liver cirrhosis, and pancreatitis), the central nervous system (cognitive deficits, severe memory impairment degenerative changes in the cerebellum, and ethanol-induced persisting amnesiac disorder in which the ability to encode new memory is severely impaired) and the cardiovascular system (hypertension, cardiomyopathy high levels of triglycerides and low-density lipoprotein cholesterol). Individuals with ethanol dependence or addiction exhibit symptoms and physical changes including dyspepsia, nausea, bloating, esophageal varices, hemorrhoids, tremor, unsteady gait, insomnia, erectile dysfunction, decreased testicular size, feminizing effects associated with reduced testosterone levels, spontaneous abortion, and fetal alcohol syndrome. Symptoms associated with ethanol cessation or withdrawal include nausea, vomiting, gastritis, hematemises, dry mouth, puffy blotchy complexion, and peripheral edema.

Other well known addictive substances are psychostimulants. Abuse of said drugs may induce tolerance and/or dependence. Withdrawal symptoms from the cessation of psychostimulants use vary greatly in intensity depending on numerous factors including the dose of the drug used et cetera.

The compound 1-[2-(4-(3-trifluoromethyl-phenyl)piperazin-1-yl)ethyl]-2,3-dihydro-1H-benzimidazol-2-one (flibanserin) is disclosed in form of its hydrochloride salt in European Patent Application EP-A-526434 and has the following chemical structure:

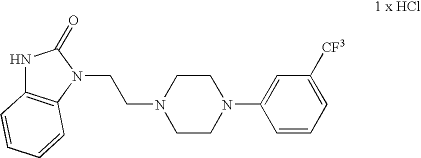

Flibanserin shows affinity for the 5-HT_{1A} and 5-HT₂-receptor. It is therefore a promising therapeutic agent for the treatment of a variety of diseases, for instance depression, schizophrenia, and anxiety.

It can be shown that flibanserin, optionally in form of the free base, the pharmacologically acceptable acid addition salts and/or optionally in form of the hydrates and/or solvates thereof can be used in the treatment of drug addiction.

Accordingly, the instant invention relates to a method for the treatment of drug addiction comprising the administration of a therapeutically effective amount of flibanserin, optionally in form of the free base, the pharmacologically acceptable acid addition salts and/or optionally in form of the hydrates and/or solvates thereof to a mammal suffering from drug addiction.

In a preferred embodiment, the invention relates to a method for the treatment of drug addiction, wherein the drug is selected from the group consisting of ethanol, nicotine and psychostimulants, comprising the administration of a therapeutically effective amount of flibanserin, optionally in form of the free base, the pharmacologically acceptable acid addition salts and/or optionally in form of the hydrates and/or solvates thereof to a mammal suffering from drug addiction.

In another preferred embodiment, the invention relates to a method of ameliorating or eliminating effects of addiction to a drug of abuse in a mammal suffering from drug addiction, comprising the administration of a therapeutically effective amount of flibanserin optionally in form of the free base, the pharmacologically acceptable acid addition salts and/or optionally in form of the hydrates and/or solvates thereof, wherein said administration is in an amount sufficient to reduce drug dependency characteristics.

In another preferred embodiment, the invention relates to a method of ameliorating or eliminating effects of addiction to nicotine, ethanol and psychostimulants in an mammal suffering from drug addiction, comprising the administration of a therapeutically effective amount of flibanserin, optionally in form of the free base, the pharmacologically acceptable acid addition salts and/or optionally in form of the hydrates and/or solvates thereof, wherein said administration is in an amount sufficient to reduce nicotine, ethanol and psychostimulant dependency characteristics.

In another preferred embodiment, the invention relates to a method for changing addiction-related behavior of a mammal suffering from drug addiction comprising the administration of a therapeutically effective amount of flibanserin, optionally in form of the free base, the pharmacologically acceptable acid addition salts and/or optionally in form of the hydrates and/or solvates thereof, wherein said administration is in an amount sufficient to diminish, inhibit or eliminate behavior associated with craving or use of said drug of abuse.

In another preferred embodiment, the invention relates to a method for changing addiction-related behavior of a mammal suffering from drug addiction, wherein the drug is selected from nicotine, ethanol and psychostimulants comprising the administration of a therapeutically effective amount of flibanserin, optionally in form of the free base, the pharmacologically acceptable acid addition salts and/or optionally in form of the hydrates and/or solvates thereof, wherein said administration is in an amount sufficient to diminish, inhibit or eliminate behavior associated with craving or use of said drug of abuse.

In another preferred embodiment, the invention relates to a method of alleviating or eliminating withdrawal symptoms in a mammal suffering from drug addiction, comprising the administration of a therapeutically effective amount of flibanserin, optionally in form of the free base, the pharmacologically acceptable acid addition salts and/or optionally in form of the hydrates and/or solvates thereof, wherein said administration is in an amount sufficient to reduce withdrawal symptoms.

In another preferred embodiment, the invention relates to a method of alleviating or eliminating withdrawal symptoms in a mammal suffering from drug addiction, wherein the drug is selected from nicotine, ethanol and psychostimulants comprising the administration of a therapeutically effective amount of flibanserin, optionally in form of the free base, the pharmacologically acceptable acid addition salts and/or optionally in form of the hydrates and/or solvates thereof, wherein said administration is in an amount sufficient to reduce or eliminate withdrawal symptoms.

Furthermore, the instant invention relates to the use of flibanserin optionally in form of the free base, the pharmacologically acceptable acid addition salts and/or optionally in form of the hydrates and/or solvates thereof for the manufacture of a medicament for the treatment of a mammal suffering from the above mentioned conditions.

In addition, the method of the present invention can be used for treating individuals addicted to a combination of drugs of abuse. For example, the mammal may be addicted to ethanol and nicotine, in which case the present invention is particularly suited for changing addiction-related behavior of the mammal by administering an effective amount of flibanserin, optionally in form of the free base, the pharmacologically acceptable acid addition salts and/or optionally in form of the hydrates and/or solvates thereof.

Examples for the psychostimulants mentioned above and below include but are not limited to amphetamine, dextroamphetamine, methamphetamine, phenmetrazine, diethylpropion, methylphenidate, cocaine, phencyclidine and pharmaceutically acceptable salts thereof.

The term withdrawal symptoms within the present invention means conditions such as anxiety, agitation, insomnia, amotivational state, depression etc.

As used herein, addiction-related behavior means behavior resulting from compulsive substance use and is characterized by apparent total dependency on the substance. Symptomatic of the behavior is (i) overwhelming involvement with the use of the drug, (ii) the securing of its supply, and (iii) a high probability of relapse after withdrawal.

For example, a cocaine user experiences three stages of drug effects. The first, acute intoxication, is euphoric, marked by decreased anxiety, enhanced self-confidence and sexual appetite, and may be marred by sexual indiscretions, irresponsible spending, and accidents attributable to reckless behavior. The second stage replaces euphoria by anxiety, fatigue, irritability and depression. Some users have committed suicide during this period. Finally, the third stage is a time of limited ability to derive pleasure from normal activities and of craving for the euphoric effects of cocaine which leads to use of this drug. As related to cocaine users, addiction-related behavior includes behavior associated with all three stages of drug effects.

Compulsive drug use includes three independent components: tolerance, psychological dependence and physical dependence. Tolerance produces a need to increase the dose of the drug after several administration in order to achieve the same magnitude of effect. Physical dependence is an adaptive state produced by repeated drug administration and which manifests itself by intense physical disturbance when drug administration is halted. Psychological dependence is a condition characterized by an intense drive, craving or use for a drug whose effects the user feels are necessary for a sense of well being.

Based on the foregoing definitions, as used herein “dependency characteristics” include all characteristics associated with compulsive drug use, characteristics that can be affected by biochemical composition of the host, physical and psychological properties of the host.

Mammals include, for example, humans, baboons and other primates, as well as pet animals such as dogs and cats, laboratory animals such as rats and mice, and farm animals such as horses, sheep, and cows, preferably humans.

As already mentioned above, flibanserin may be used in form of the free base, optionally in form of its pharmaceutically acceptable acid addition salts and/or optionally in form of the hydrates and/or solvates thereof. Suitable acid addition salts include for example those of the acids selected from, succinic acid, hydrobromic acid, acetic acid, fumaric acid, maleic acid, methanesulphonic acid, lactic acid, phosphoric acid, hydrochloric acid, sulphuric acid, tartaric acid and citric acid. Mixtures of the abovementioned acid addition salts may also be used. From the aforementioned acid addition salts the hydrochloride and the hydrobromide, particularly the hydrochloride, are preferred. If flibanserin is used in form of the free base, it is preferably used in form of flibanserin polymorph A as disclosed in WO 03/014079.

Flibanserin, optionally in form of the free base, the pharmacologically acceptable acid addition salts and/or optionally in form of the hydrates and/or solvates thereof, may be incorporated into the conventional pharmaceutical preparation in solid, liquid or spray form. The composition may, for example, be presented in a form suitable for oral, rectal, parenteral administration or for nasal inhalation: preferred forms includes for example, capsules, tablets, coated tablets, ampoules, suppositories and nasal spray.

The active ingredient may be incorporated in excipients or carriers conventionally used in pharmaceutical compositions such as, for example, talc, arabic gum, lactose, gelatine, magnesium stearate, corn starch, acqueous or non acqueous vehicles, polyvynil pyrrolidone, semisynthetic glicerides of fatty acids, benzalconium chloride, sodium phosphate, EDTA, polysorbate 80. The compositions are advantageously formulated in dosage units, each dosage unit being adapted to supply a single dose of the active ingredient. The dosage range applicable per day is between 0.1 to 400, preferably between 1.0 to 300, more preferably between 2 to 200 mg.

Each dosage unit may conveniently contain from 0.01 mg to 100 mg, preferably from 0.1 to 50 mg.

Suitable tablets may be obtained, for example, by mixing the active substance(s) (flibanserin base and/or salts) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g of. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g of. with the addition of preservatives such as p-hydroxybenzoates, or stabilisers such as alkali metal salts of ethylenediamine tetraacetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

The Examples which follow illustrate the present invention without restricting its scope:

### EXAMPLES OF PHARMACEUTICAL FORMULATIONS

| | | |
|---|---|---|
| A) | Tablets | per tablet |

| | | |
|---|---|---|
| flibanserin hydrochloride | 100 | mg |
| lactose | 240 | mg |
| corn starch | 340 | mg |
| polyvinylpyrrolidone | 45 | mg |
| magnesium stearate | 15 | mg |
| | 740 | mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| | | |
|---|---|---|
| B) | Tablets | per tablet |

| | | |
|---|---|---|
| flibanserin hydrochloride | 80 | mg |
| corn starch | 190 | mg |
| lactose | 55 | mg |
| microcrystalline cellulose | 35 | mg |
| polyvinylpyrrolidone | 15 | mg |
| sodium-carboxymethyl starch | 23 | mg |
| magnesium stearate | 2 | mg |
| | 400 | mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

| | | |
|---|---|---|
| C) | Coated tablets | per coated tablet |

| | | |
|---|---|---|
| flibanserin hydrochloride | 5 | mg |
| corn starch | 41.5 | mg |
| lactose | 30 | mg |
| polyvinylpyrrolidone | 3 | mg |
| magnesium stearate | 0.5 | mg |
| | 80 | mg |

The active substance, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in known manner with a covering consisting essentially of sugar and talc. The finished coated tablets are polished with wax.

| | | |
|---|---|---|
| D) | Capsules | per capsule |

| | | |
|---|---|---|
| flibanserin hydrochloride | 150 | mg |
| Corn starch | 268.5 | mg |
| Magnesium stearate | 1.5 | mg |
| | 420 | mg |

The substance and corn starch are mixed and moistened with water. The moist mass is screened and dried. The dry granules are screened and mixed with magnesium stearate. The finished mixture is packed into size 1 hard gelatine capsules.

| | |
|---|---|
| E) | Ampoule solution |

| | | |
|---|---|---|
| flibanserin hydrochloride | 50 | mg |
| sodium chloride | 50 | mg |
| water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion.

| | |
|---|---|
| F) | Suppositories |

| | | |
|---|---|---|
| flibanserin hydrochloride | 50 | mg |
| solid fat | 1650 | mg |
| | 1700 | mg |

The hard fat is melted. At 40° C. the ground active substance is homogeneously dispersed. It is cooled to 38° C. and poured into slightly chilled suppository moulds.

In a particular preferred embodiment of the instant invention, flibanserin is administered in form of specific film coated tablets. Examples of these preferred formulations are listed below. The film coated tablets listed below can be manufactured according to procedures known in the art (see hereto WO 03/097058).

G) Film Coated Tablet

| | |
|---|---|
| Constituents | mg/tablet |

Core

| | |
|---|---|
| Flibanserin (polymorph A) | 25.000 |
| Lactose monohydrate | 71.720 |
| Microcrystalline cellulose | 23.905 |
| HPMC (Methocel E5) | 1.250 |
| Carboxymethylcellulose sodium | 2.500 |
| Magnesium stearate | 0.625 |

Coating

| | |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 128.000 |

H) Film Coated Tablet

| | |
|---|---|
| Constituents | mg/tablet |

Core

| | |
|---|---|
| Flibanserin (polymorph A) | 50.000 |
| Lactose monohydrate | 143.440 |
| Microcrystalline cellulose | 47.810 |
| HPMC (e.g. Pharmacoat 606) | 2.500 |
| Carboxymethylcellulose sodium | 5.000 |
| Magnesium stearate | 1.250 |

Coating

| | |
|---|---|
| HPMC (e.g. Pharmacoat 606) | 2.400 |
| Polyethylene Glycol 6000 | 0.700 |
| Titanium dioxide | 1.000 |
| Talc | 0.857 |
| Iron oxide red | 0.043 |
| Total Film coated tablet | 255.000 |

I) Film Coated Tablet

| | |
|---|---|
| Constituents | mg/tablet |

Core

| | |
|---|---|
| Flibanserin (polymorph A) | 100.000 |
| Lactose monohydrate | 171.080 |
| Microcrystalline cellulose | 57.020 |
| HPMC (e.g. Methocel E5) | 3.400 |
| Carboxymethylcellulose sodium | 6.800 |
| Magnesium stearate | 1.700 |

Coating

| | |
|---|---|
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated tablet | 347.000 |

J) Film Coated Tablet

| | |
|---|---|
| Constituents | mg/tablet |

Core

| | |
|---|---|
| Flibanserin (polymorph A) | 2.000 |
| Dibasic Calciumphosphate, anhydrous | 61.010 |
| Microcrystalline cellulose | 61.010 |
| HPMC (Methocel E5) | 1.950 |
| Carboxymethylcellulose sodium | 2.600 |
| Colloidal silicon dioxide | 0.650 |
| Magnesium stearate | 0.780 |

Coating

| | |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 133.000 |

K) Film Coated Tablet

| | |
|---|---|
| Constituents | mg/tablet |

Core

| | |
|---|---|
| Flibanserin (polymorph A) | 100.000 |
| Dibasic Calciumphosphate, anhydrous | 69.750 |
| Microcrystalline cellulose | 69.750 |
| HPMC (e.g. Methocel E5) | 2.750 |
| Carboxymethylcellulose sodium | 5.000 |
| Colloidal silicon dioxide | 1.250 |
| Magnesium stearate | 1.500 |

Coating

| | |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 255.000 |

L) Film Coated Tablet

| | |
|---|---|
| Constituents | mg/tablet |

Core

| | |
|---|---|
| Flibanserin (polymorph A) | 20.000 |
| Lactose monohydrate | 130.000 |
| Microcrystalline cellulose | 43.100 |
| Hydroxypropyl Cellulose (e.g. Klucel LF) | 1.900 |
| Sodium Starch Glycolate | 4.000 |
| Magnesium stearate | 1.000 |

Coating

| | |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 205.000 |

What is claimed is:

1) A method for the treatment of drug addiction comprising the administration of a therapeutically effective amount of flibanserin, in form of the free base, a pharmacologically acceptable acid addition salt or in form of a hydrate or a solvate thereof, or a combination thereof, to a mammal suffering from drug addiction.

2) A method of ameliorating or eliminating effects of addiction to a drug of abuse in a mammal suffering from drug addiction, comprising the administration of a therapeutically effective amount of flibanserin, in form of the free base, a pharmacologically acceptable acid addition salt or in form of a hydrate or a solvate thereof, or a combination thereof, wherein said administration is in an amount sufficient to reduce drug dependency characteristics.

3) A method for changing addiction-related behavior of a mammal suffering from drug addiction comprising the administration of a therapeutically effective amount of flibanserin, in form of the free base, a pharmacologically acceptable acid addition salt or in form of a hydrate or a solvate thereof, or a combination thereof, wherein said administration is in an amount sufficient to diminish, inhibit or eliminate behavior associated with craving or use of said drug of abuse.

4) A method for alleviating or eliminating withdrawal symptoms in a mammal suffering from drug addiction, comprising the administration of a therapeutically effective amount of flibanserin, in form of the free base, a pharmacologically acceptable acid addition salt or in form of a hydrate or a solvate thereof, or a combination thereof, wherein said administration is in an amount sufficient to reduce withdrawal symptoms.

5) The method according to claim 1 characterized in that the drug is selected from nicotine, ethanol or a psychostimulant.

6) The method according to claim 1, characterized in that flibanserin is applied in form of a pharmaceutically acceptable acid addition salt selected from the salts formed by the acids selected from, succinic acid, hydrobromic acid, acetic acid, fumaric acid, maleic acid, methanesulphonic acid, lactic acid, phosphoric acid, hydrochloric acid, sulphuric acid, tartaric acid, citric acid, and mixtures thereof.

7) The method according to claim 6, characterized in that flibanserin is applied in form of flibanserin polymorph A.

8) The method accoridng to claim 7, characterized in that flibanserin is applied in a dosage range between about 0.1 to about 400 mg per day.
